(19) United States
(12) Patent Application Publication
(10) Pub. No.: US 2004/0010028 A1
(43) Pub. Date: Jan. 15, 2004

(54) Self-assembled nanostructures with macroscopic polar order

(76) Inventor: Colin Peter Nuckolls, New York, NY (US)

Correspondence Address: BAKER & BOTTS, 30 ROCKEFELLER PLAZA, NEW YORK, NY, 10112

(21) Appl. No.: 10/381,680

(22) PCT Filed: Oct. 30, 2001
(86) PCT No.: PCT/US01/48479

(51) Int. Cl. A61K 31/409

(52) U.S. Cl. 514/410

(57) ABSTRACT

A three-dimensional molecular array on one of a conductor and semiconductor surface is disclosed. The array comprises at least one columnar stack comprising a plurality of substituted aromatic rings, wherein the aromatic rings of each columnar stack lie about parallel to the surface, wherein the columnar stack comprises a plurality of hydrogen bonds between substituents of different rings. Each stack may include aromatic amides in which the amido group is not coplanar with the aromatic core. A method for the synthesis of such aromatic amides is also disclosed. The aromatic amides may also be used to prepare pyro-ferro-, and piezo electric devices, as well as nonlinear optical devices and conductors, which comprise the columnar stacks described.

### CROSS-REFERENCE TO RELATED APPLICATION

This application claims priority from U.S. Provisional Application Ser. No. 60/244,320, filed Oct. 30, 2000.

### BACKGROUND OF THE INVENTION

1. Field of the Invention

The invention is directed at molecular systems comprising aromatic rings which self-assemble to form a columnar nanostructure. In particular, the invention is directed at molecular arrays which are self-assembled through hydrogen bond formation.

2. Background Information

Synthesis and molecular recognition can be used to create novel materials and structures whose emergent or amplified properties arise from self-assembly, as discussed in Lawrence, D. S., Jiang, T., and Levett, M., “Self-Assembling Supramolecular Complexes, ” Chemical Reviews, Vol. 97 (1997), pp. 1647-68. This approach is believed to be instrumental in the continued miniaturization of electronic and optic components into the nanoscale regime. Organic molecules are particularly attractive because their macroscopic properties can be fine-tuned at the molecular level through organic synthesis.

Organic molecules that spontaneously self-organize into polar structures are rare but highly desirable because they do not require electric poling fields and still can provide useful piezoelectric, pyroelectric, and ferroelectric responses. Polar ordering has been demonstrated for organic materials. Examples include nearest neighbor interactions in chiral columns, as disclosed in Bock, H., and Helfrich, W., “Ferroelectrically Switchable Columnar Liquid Crystal, ” Liquid Crystals, Vol. 12 (1992), pp. 697-703, and grafting of dipolar polypeptide strands from surfaces, as discussed in Jaworek, T., Neher, D., Wegner, G., Wieringa, R. H., and Schouten, A. J., “Electromechanical Properties of an Ultrathin Layer of Directionally Aligned Helical Polypeptides,” Science, Vol. 279 (1998), pp. 57-60.

One class of structures that has shown promise in electronic and optic applications are discotic liquid crystals, fluid columnar structures that are formed by stacks of aromatic molecules surrounded by alkyl chains. A review of the field is given in Chandrasekhar, S., Prasad, S., and Krishna, “Recent developments in discotic liquid crystals,” Contemporary Physics, Vol. 40 (1999), pp. 237-245. This superstructural motif has been likened to a “nanowire” with a hydrocarbon sheathed around a π-stacked core (FIG. 1) by Chandrasekhar, S., “Columnar, Discotic Nematic and Lamellar Liquid Crystals: their Structures and Physical Properties,” Handbook of liquid Crystals, Vol. 2B (1998), pp. 749-780. Indeed, once assembled these molecules have been shown to delocalize charges through their centers. Numerous types of molecules have been shown to organize in this manner, as demonstrated by Chandrasekhar, S., and Ranganath, G. S., “Discotic Liquid Crystals,” Rep. Prog. Phys., Vol. 53 (1990), pp. 57-84; Destrade, C., Foucher, P., Gasparoux, H., Nguyen H. T., Levelut, A. M., and Malthete, J., “Disk-like Mesogen Polymorphism,” Mol. Cryst. Liq. Cryst., Vol. 706 (1984), pp. 121-46; Simon, J., and Bassoul, P., “Phthalocyanines: Properties and Applications,” New York, Leznoff, C. C., Lever, A. B. P., Eds., 1989, Vol. 2, Chapter 6; and Serrette, A. G., Lai, C. K., and Swager, T. M., “Complementary Shapes in Columnar Liquid Crystals: Structural Control in Homo- and Heteronuclear Bimetallic Assemblies,” Chem. Mater., Vol. 6 (1994), pp. 2252-68.

Even though these structures are held together only by weak and nondirectional forces, several research groups have succeeding in doping their structures with either acceptor or donor impurities, and some of these studies produced charge carrier mobilities approaching useful values. The nanoscale columns are plastic, self-repairing, one-dimensional semiconductors.

Defects and irregularities are believed to limit the charge carrier mobilities in these self-assembled stacks. Moreover, in traditional discotic liquid crystals the columnar structures are continually breaking and reforming on a time-scale that is on the order of 10⁻⁵ s, as shown by Dong, R. Y., Goldfarb, D., Moseley, M. E., Luz, Z., and Zimmermann, H., “Translational Diffusion in Discotic Mesophases Studied by the Nuclear Magnetic Resonance Pulsed Field Gradient Method, ” Journal of Physical Chemistry, Vol. 88 (1984), pp. 3148-52. Just as is the case with traditional semiconductor materials, it is believed that creating a more regular material will create greater transport capacity. Support for this statement is provided by the recent discovery of low temperature superconductivity in cleaved, single crystals of pentacene by Schon, J. H., Kloc, Ch., and Batlogg, B., “Superconductivity in Molecular Crystals Induced by Charge Injection,” Nature, Vol. 406 (2000), pp. 702-704. If organic π-surfaces are appropriately arranged, their potential as conductors appears to be limitless. Furthermore, it is known in the art that structures having polar order, in addition to conductance, also exhibit piezoelectric, ferroelectric, pyroelectric, and non-linear optical properties as shown in the following references (herein incorporated by reference): Burland, D. M., Miller, R. D., and Walsh, C. A., “Second-order Nonlinearity in Poled-Polymer Systems, ” Chemical Reviews, Vol. 94 (1994), pp. 31-75; Pralle, M. U., Urayama, K., Tew, G. N., Neher, D., Wegner, G., and Stupp, S. I., “Piezoelectricity in Polar Supramolecular Materials,” Angewandte Chemie, International Edition in English, Vol. 39(8) (2000), pp. 1486-1489; and “Electromechanical Properties of an Ultrathin Layer of Directionally Aligned Helical Polypeptides,” by Jaworek and co-workers, discussed above. Conductance in a system of stacked rings is along the axis of the stack, as shown in the following references (herein incorporated by reference): Van de Craats, A. M., Warman, J. M., Fechtenkotter, A., Brand, J. D., Harbison, M. A., and Mullen, K., Advanced Materials, Vol. 11 (1999), pp. 1469-72; Chandrasekhar, S., and Prasad, S. K., Contemporary Physics, Vol. 40 (1999), pp. 237-45; and Boden, N., Bushby, R. J., Clements, J., and Movaghar, B. Journal of Materials Chemistry, Vol. 9 (1999), pp. 2081-86.

Incorporating well-defined hydrogen bonding functionality into the core of the aromatic structure is believed to slow the time-scale for the breaking and reforming of the columnar structures. This concept was first elucidated in Matsunaga, Y., Nakayasu, Y., Sakai, S., and Yonenaga, M., “Liquid Crystal Phases Exhibited by N,N′,N″-trialkyl-1,3,5-benzenetricarboxamides,” Mol. Cryst. Liq. Cryst., Vol. 141 (1986), pp. 327-33, which demonstrated that benzene rings that are substituted with the three meta-disposed secondary amides stack to form columnar, liquid-crystalline phases. Subsequent derivatives of these structures primarily involve substitution of the amide nitrogens with functionality of increasing complexity, as disclosed in Palmans, A. R. A., Vekemans, J. A. J. M., Havinga, E. E., and Meijer, E. W., “Sergeants-and-soldiers Principle in Chiral Columnar Stacks of Disk-shaped Molecules with C3 Symmetry, ” Angewandte Chemie, International Edition in English, Vol, 36 (1997), pp. 2648-51; Brunsveld, L., Zhang, H., Glasbeek, M., Vekemans, J. A. J. M., and Meijer, E. W., “Hierarchical Growth of Chiral Self-Assembled Structures in Protic Media,” J. Am. Chem. Soc., Vol. 122 (2000), pp. 6175-82; Palmans, A. R. A., Vekemans, J. A. J. M., Fischer, H., Hikmet, R. A., and Meijer, E. W., “Extended-core Discotic Liquid Crystals Based on the Intramolecular H-bonding in N-acylated 2,2′-bipyridine-3,3′-diamine Moieties,” Chemistry—a European Journal, Vol. 3 (1997), pp. 300-307; and Yasuda, Y., Iishi, E., Inada, H., and Shirota, Y., “Novel Low-molecular-weight Organic Gels: N,N′,N″-tristearyltrimesamide/Organic Solvent System,” Chemistry Letters, vol. 7 (1996), pp. 575-576.

There exist only a limited numbers of examples of discotic liquid crystals assembling from derivatized surfaces. In Hiesgen, R., Schonherr, H., Kumar, S., Ringsdorf, H., and Meissner, D., “Scanning Tunneling Microscopy Investigation of Tricycloquinazoline Liquid Crystals on Gold,” Thin Solid Films, Vol. 358 (2000), pp. 241-249, alkylthiols substituents on a tricycloquinazoline discotic core were used to orient structures on gold. In Vauchier, C., Zarin, A., Le Barny, P., Dubois, J. C., and Billard, J., “Orientation of Discotic Mesophases,” Molecular Crystals and Liquid Crystals, vol. 66 (1981), pp. 423-33, noncovalent interactions with derivatized aromatics were used to coat glass surfaces.

There is, however, a lack in the art of columnar structures on a conductor or semiconductor surface having one or more stacks of substituted benzene rings lying parallel to the surface in which each of the substituents of different rings within each stack form hydrogen bonds.

### SUMMARY OF THE INVENTION

Accordingly, it is an object of this invention to provide a three-dimensional molecular array on one of a conductor and a semiconductor surface, the array comprising at least one columnar stack comprising a plurality of substituted aromatic rings, wherein the aromatic rings of each columnar stack lie about parallel to the surface and the columnar stack comprises a plurality of hydrogen bonds between substituents of different rings.

It is another object of this invention to provide aromatic amides in which the amido group is not coplanar with the aromatic core, and a method for the synthesis of such aromatic amides.

It is another object of the invention to provide a method for the preparation of a molecular array on one of a conductor and a semiconductor surface, the molecular array comprising at least one columnar stack, the method comprising (a) coating onto one of a conductor and a semiconductor surface a surface template compound comprising substituted aromatic rings to form a surface template molecular monolayer; and (b) coating onto the surface template monolayer a second compound comprising substituted aromatic rings to form at least one columnar stack comprising the substituted aromatic rings of the second compound, wherein the aromatic rings of each columnar stack and of the surface template molecules lie about parallel to the surface and the columnar stack comprises a plurality of hydrogen bonds between substituents of different rings.

It is another object of this invention to provide a one-dimensional conductor on one of a conductor and a semiconductor surface, wherein conductance of the one-dimensional conductor is in a direction about perpendicular to one of the conductor and the semiconductor surface, wherein the one-dimensional conductor is prepared by the method comprising (a) providing a solution of a compound comprising a substituted aromatic ring; (b) providing two electrodes having parallel surfaces in contact with the solution, wherein each electrode is one of a conductor and semiconductor; and (c) applying an electric potential between the electrodes, thereby forming a plurality of columnar stacks each comprising a plurality of substituted aromatic rings, wherein the aromatic rings of each one of the columnar stacks lie about parallel to the surface of each one of the electrodes, wherein each one of the columnar stacks comprises a plurality of hydrogen bonds between substituents of different rings and each one of the columnar stacks has a respective dipole moment aligned between the two electrodes.

It is another object of this invention to provide a device having at least one of piezoelectric, ferroelectric, pyroelectric, and non-linear optical properties, wherein the device is prepared by the method comprising (a) coating onto one of a conductor and a semiconductor surface a surface template compound comprising substituted aromatic rings to form a surface template molecular monolayer, (b) rinsing and drying the surface; (c) coating onto the monolayer a second compound comprising substituted aromatic rings to form a second layer comprising at least one columnar stack of the aromatic rings of the second compound, wherein the aromatic rings of each one of the at least one columnar stack and of the surface template molecules lie about parallel to the surface, wherein each one of the at least one columnar stack comprises a plurality of hydrogen bonds between substituents of different rings; and (d) evaporating onto the second layer another conductive layer so as to provide another conductive surface.

The molecular arrays of the invention exhibit polar order since each stack is oriented about perpendicular to the surface to maximize hydrogen bonding within the stack while maintaining the aromatic rings about parallel to the surface. As discussed above, it is known in the art that structures having polar order exhibit piezoelectric, ferroelectric, pyroelectric, conductive, and non-linear optical properties. Accordingly, the molecular arrays of the invention have at least one of piezoelectric, ferroelectric, pyroelectric, conductive, and non-linear optical properties. At least one of piezoelectric, ferroelectric, pyroelectric, conductive, and non-linear optical properties of the device of the invention is due to the formation of polar order molecular arrays in the step of the method wherein the second compound comprising substituted aromatic rings is coated onto the molecular monolayer of the surface template compound.

By developing the novel, crowded rings of this invention, access is gained to macroscopically polar structures whose arrangement of dipoles (in the superstructure) is similar to that of helically wound polybenzylglutamates “grafted-from” amine containing surfaces as discussed in “Electromechanical Properties of an Ultrathin Layer of Directionally Aligned Helical Polypeptides,” discussed above. This study demonstrated that these polymers organized into colinear helices having aligned amide dipoles gives rise to a piezoelectric response. Although the magnitude in these systems was nearly equal to that of material used in commercial piezoelectric devices, it is believed that their piezoelectric response is limited by the large mechanical tensor along the peptide backbone.

Furthermore, it is known in the art that aromatic amides can be used as molecular recognition devices. Accordingly, the aromatic amides and sulfinamides of the invention are useful as molecular recognition devices as well as being able to form columnar stacks.

### BRIEF DESCRIPTION OF THE FIGURES

FIG. 1 shows a schematic illustration of discotic liquid crystals.

FIG. 2 shows a three-dimensional molecular structure of a single one molecule wide columnar stack.

FIG. 3 shows a schematic illustration of cooperative hydrogen bonding and π-stacking.

FIG. 4 shows a schematic illustration of triple hydrogen-bond surface templates.

FIG. 5 shows a schematic illustration of stacking on a surface template.

FIG. 6 shows a schematic illustration of polar order in columnar stacks.

FIG. 7 shows a proposed mechanism for constriction/elongation of columns.

FIG. 8 shows (a) a plastic crystalline texture plot and X-ray diffraction of 1a and (b) a liquid crystalline texture plot and and X-ray diffraction of 1d.

FIG. 9 shows a schematic piezoelectric and ferroelectric nanoscale device.

FIG. 10 shows a schematic nanoscale self-assembled one-dimensional conductor.

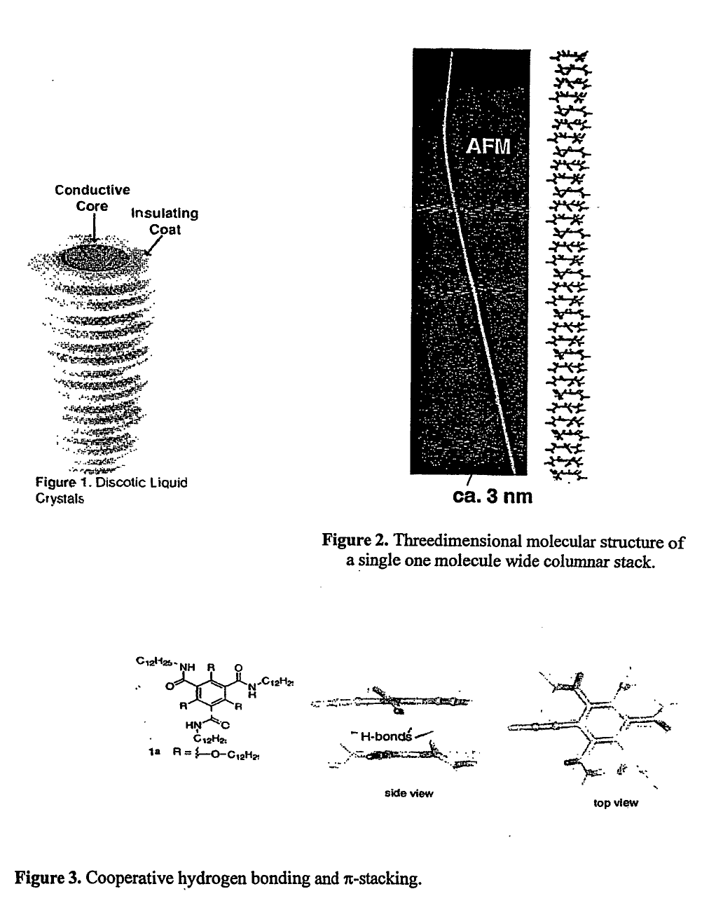

### DETAILED DESCRIPTION OF THE INVENTION

In an exemplary embodiment of the present invention, the surface is a conductor surface or a semiconductor surface and the columnar stack is a one-dimensional conductor. Since the aromatic rings are stacked about parallel to the surface and the hydrogen bonds are formed between successive aromatic rings, the columnar stack is about perpendicular to the surface of the conductor or semiconductor. Since conductance in a system of stacked rings is along the axis of the stack, conductance in each stack of the present invention is in a direction about perpendicular to the conductor or semiconductor surface. Similarly, conductance in each molecular array comprising at least one stack of the present invention is in a direction about perpendicular to the surface. Similarly, conductance in a conductor of the present invention is in a direction about perpendicular to the surface.

In another exemplary embodiment, each columnar stack of the molecular array is chiral. The chirality of the columnar stack is preferably due to the presence of chiral substituents on the aromatic rings comprising the stack.

In yet another exemplary embodiment, the aromatic rings are not covalently bound to each other.

Advantageously, the columnar stacks comprise substituted aromatic derivatives having a hydroxy group or an amino group lying outside the plane of the aromatic ring.

In a particularly advantageous exemplary embodiment, each aromatic ring in the stack comprises a first substituent in each of the 2, 4, and 6 positions of the ring, wherein the first substituent is not hydrogen, and a second substituent in each of the 1, 3, and 5 positions of the ring, wherein the second substituent is selected from the group consisting of a carboxylic group and a group having the structural formula

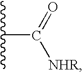

wherein R is selected from the group consisting of hydroxy, substituted and unsubstituted alkoxy, substituted and unsubstituted alkyl, substituted and unsubstituted aryl, substituted and unsubstituted acyl, and

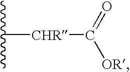

wherein R″ is selected from the group consisting of hydrogen, a substituted alkyl and an unsubstituted alkyl, and R′ is selected from the group consisting of hydrogen, a substituted alkyl and an unsubstituted alkyl.

In the exemplary embodiments described herein, R″ is selected from hydrogen, methyl, thiomethyl, and isobutyl.

In still another exemplary embodiment, the first substituent is an alkoxy substituent, n—CₙH₂ₙ₊₁O—, where n is from 1 to 14, and the second substituent is selected from the group consisting of substituents I-IX below:

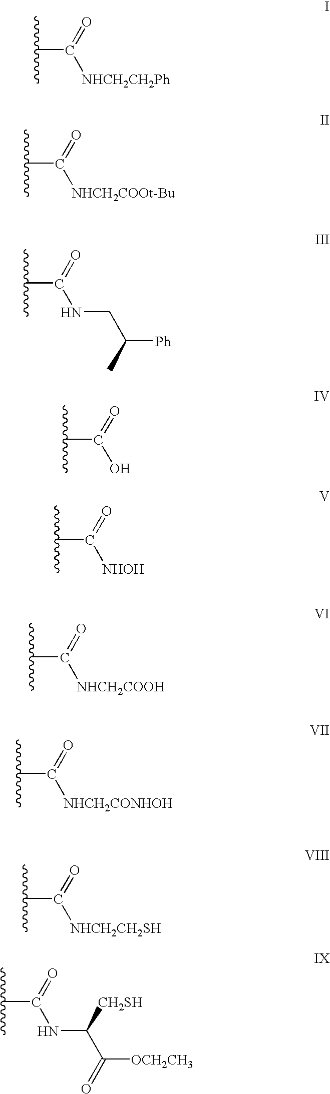

Advantageously, the first substituent is n-dodecyloxy.

It is understood that the terms substituted alkoxy, substituted alkyl, and substituted acyl signify alkoxy, alkyl, and acyl groups, respectively, in which the alkyl chains of each group include one or more susbtituents, where the substituents may be halogen, amino, mono- or di-alkylamino, hydroxy, alkoxy, alkyl, aryl, or mixtures thereof. It is understood that the term substituted aryl signifies an aryl group bearing one or more substituents, where the substituents may be halogen, amino, mono- or di-alkylamino, hydroxy, alkoxy, alkyl, aryl, or mixtures thereof.

In another particularly advantageous exemplary embodiment, each aromatic ring comprises a sulfinamide substituent in each of the 1, 3, and 5 positions of the ring, wherein the nitrogen atom in each sulfinamide substituent is bound to at least one hydrogen atom. Advantageously, each sulfinamide substituent has the same chiral configuration. Furthermore, it is advantageous that the sulfinamide substituent is selected from the group consisting of substituents X-XV below and their enantiomers:

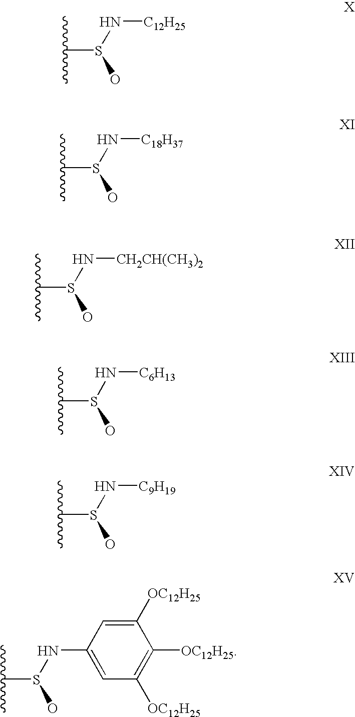

In a further exemplary embodiment of the invention, the aromatic rings forming the columnar stack may be covalently bound. Advantageously, the columnar stack consists of a single molecule, in which hydrogen bonds are intramolecular. In a still further exemplary embodiment, the columnar stack has the structure

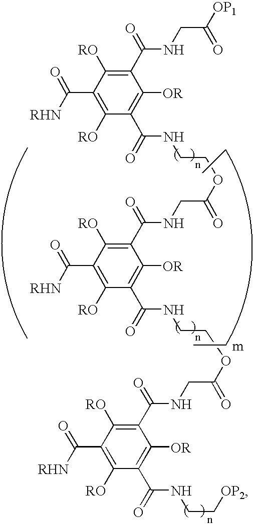

wherein n is from 5 to 10 and m is from 5 to 15.

The presence of substituents in the 2, 4, and 6 positions in the above described trialkylamides ensures that the amino group in each alkylamide substituent lies out of the plane of the aromatic ring of the molecule even before stacking occurs. Similarly, the non-planar configuration of the sulfur atom in the sulfinamide group ensures that the amino group in each sulfinamide substituent lies out of the plane of the aromatic ring of the molecule even before stacking occurs.

In another exemplary embodiment of the invention, the conductor surface is selected from the group consisting of a metal surface, a metal oxide surface, and a graphite surface.

In an alternative exemplary embodiment of the invention, the semiconductor surface is a silicon surface. The semiconductor surface may be doped with either acceptor or donor impurities using standard procedures that are known in the art.

In yet another particularly advantageous exemplary embodiment of the invention the array comprises a single one molecule wide columnar stack on one of a conductor and semiconductor surface, where the columnar stack comprises a plurality of substituted aromatic rings and the aromatic rings of each columnar stack lie about parallel to the conductor or semiconductor surface, and where the columnar stack comprises a plurality of hydrogen bonds between substituents of different rings. This embodiment of the invention is shown in FIG. 2.

Not only does the surface control the positioning of the aromatic cores; it also provides a uniform direction to the amide dipoles assembled from the surface. Thus, the materials formed from a surface template would have macroscopic polar order as a result of the parallel columnar dipoles. This property is a prerequisite for creating nanodevices having ferroelectric, piezoelectric, and pyroelectric properties.

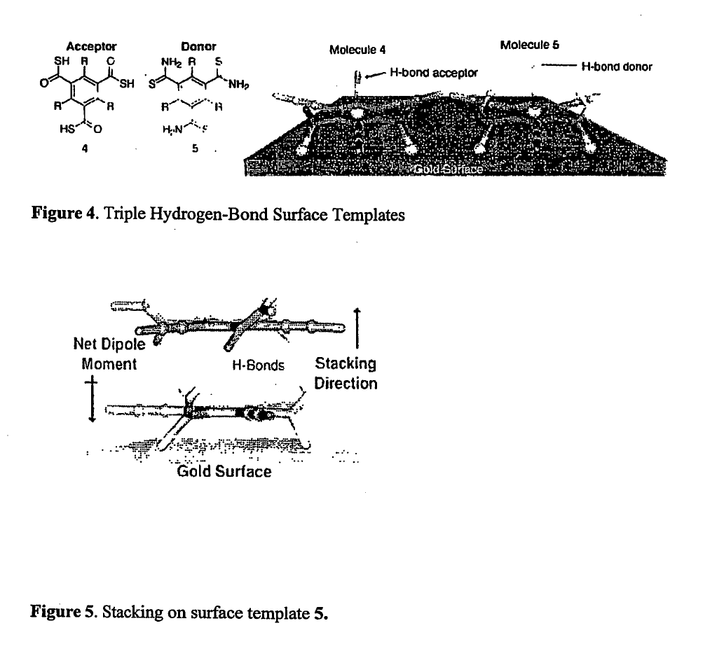

For example, as shown in FIG. 4, the thioacid functionality of surface template molecule 4 and thioamide functionality of surface template molecule 5 react with a gold surface yielding either hydrogen bond acceptor or hydrogen-bond donor groups that are directed upward, poised for hydrogen bonding. By exposing these derivatized surfaces to a complementary molecule 1, such as 1a in FIG. 3 (see also Scheme 1 below), the assembly occurs giving rise to π-stacks surrounded by insulating material—nanowires—growing from a conductive surface. For example, in the case of 5, the interaction with a complementary molecule occurs as shown in FIG. 5.

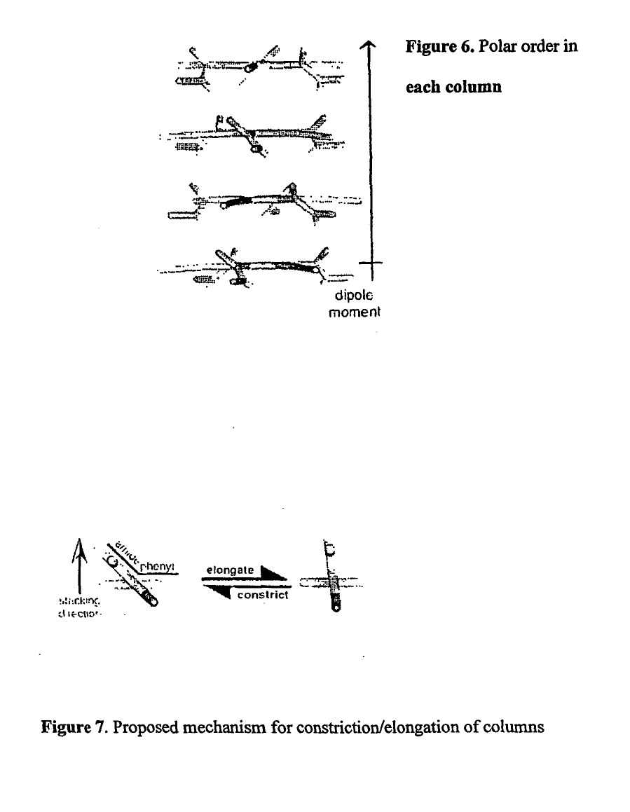

The columnar stacks exhibit polar order due to the hydrogen bonding interactions between substituents on successive rings of the stack (FIG. 6).

The molecules of the invention can maintain good hydrogen bond distances and sacrifice π-stacking by rotating their amides as shown in FIG. 7. As the piezoelectricity is known to be proportional to how easily stretched an assembly is along the polar axis, the molecular arrays of the present invention have a large piezoelectric response because they can elongate and constrict by this mechanism. In contrast, peptide helices, for example, would have to break hydrogen bonds to elongate. The relatively high energy required for this process in peptide helices is expected to lead to a lower piezoelectric response than for molecular arrays of the present invention having a comparable number of hydrogen bonds.

Shown in Scheme 1 is the synthesis of 1a-d. Each of the sidechains can be varied independently providing access to a separate class of molecules.

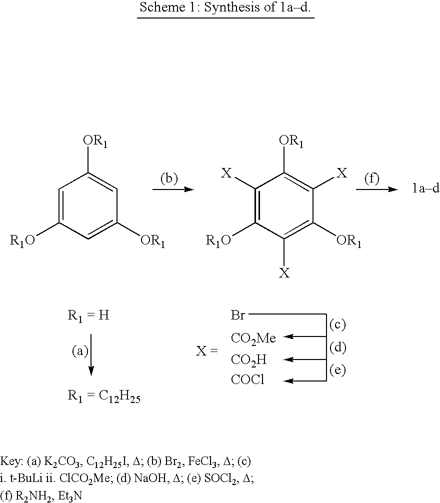

As shown in Scheme 1 above, the synthesis of structures 1 and similar structures with three amide substituents meta-disposed to three alkoxy groups began by brominating the alkylation product of phluorglucinol and 1-iodododecane by a procedure analogous to that described in Engman, L., and Hellberg, J. S. E., “A General Procedure for the Synthesis of Methylthio-, Methylseleno-and Methyltelluro-substituted Aromatic Compounds,” Journal of Organometallic Chemistry, Vol. 296 (1985), pp. 357-66 (herein incorporated by reference), where triple lithium/halogen exchange was followed by a methylchloroforrnate quench following the procedure described by Tatsuta, K., Tamura, T., and Mase, T., “The First Total Synthesis of Sideroxylonal B, ” Tetrahedron Letters, vol. 40 (1999), pp. 1925-28 (herein incorporated by reference). Saponification of the esters then yielded the key-intermediate in the synthesis, the tris-carboxylic acid. The three secondary amides of 1a-d could then be introduced in near quantitative yield by conversion to the acid chloride and reaction with dodecylamine. Details of the synthetic procedure are described in Example 1 herein. The last step, involving preparation of 1a-d from the acid chloride, can be applied to the synthesis of trialkylamides in general. R₂ in 1a-1d is n-dodecyl, phenethyl, methyl, and —CH₂—C(O)O—t—C₄H₉, respectively.

When 1 is dissolved in hexane at millimolar concentrations, a stiff gel results. Moreover these gels are birefringent when viewed with a polarized light microscope, and individual highly birefringent fibers could be seen in these samples. This is a strong indication that there is order beyond the molecular level. It is noted that spin coating from these hexanes solutions produces highly-order thin films.

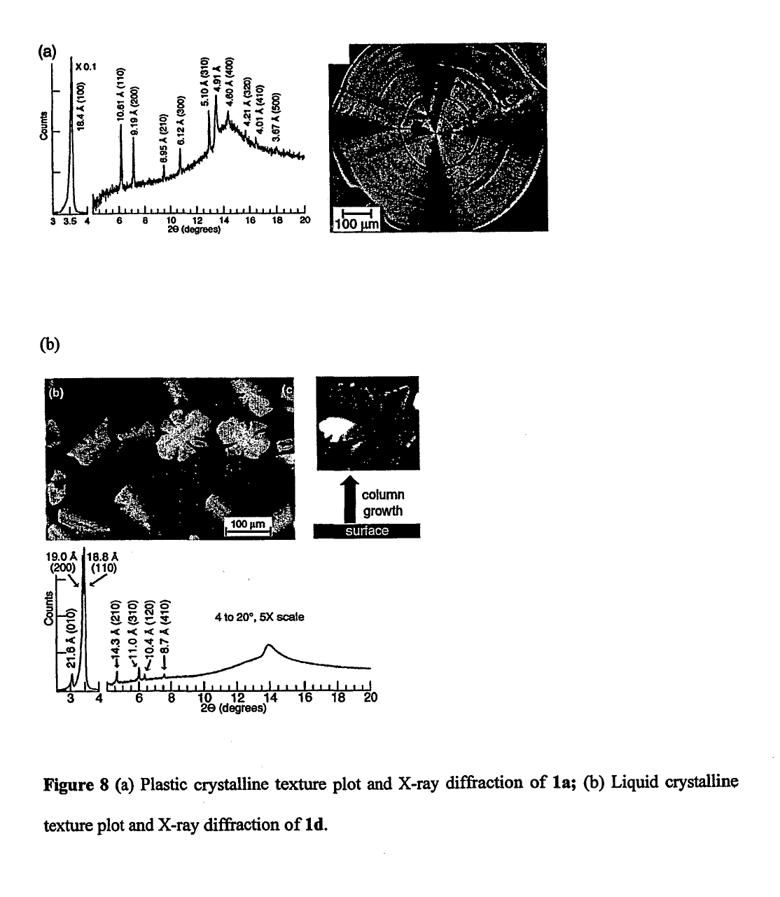

Neat samples viewed with a polarized light microscope are brightly birefringent. The viscosity of the material decreases as the temperature is raised becoming clearly fluid above ca. 145° C. although still birefringent. The material does not become an isotropic liquid until 288° C. Upon cooling, a uniform texture begins to develop. At 255° C. the sample is fluid, and the pattern shown in FIG. 8a can be deformed as pressure is applied. If the sample is cooled to room temperature, the texture is maintained although the sample is much less fluid. This phase behavior is reversible: if the samples are heated again beyond the clearing point the texture disappears only to reappear after cooling.

FIG. 8a shows a plastic crystalline texture plot and X-ray diffraction of 1a. FIG. 8b shows a liquid crystalline texture plot and X-ray diffraction of 1d.

Surface templates may include 2,4,6-trisubstituted benzenes having 1,3,5-trialkalkylamides substituents, which were prepared by the same procedure described in Example 1 herein. Surface templates preferably include 2,4,6-trisubstituted benzenes having a substituent in each of the 1, 3 and 5 positions of the ring selected from the group consisting of substituents I-IX described above. Advantageously, the surface template is structure 3 below. The surface templates having general formulas 4 and 5 below can also be used. 4 was prepared from a trialkyamide analogous to 1a-d but with R₂=H and reacting it with an excess of Lawesson's reagent. 5 was prepared by reacting the triacid chloride (an intermediate in the synthesis of 1 discussed above) with hydrogen sulfide gas that was bubbled through the solution of 5.

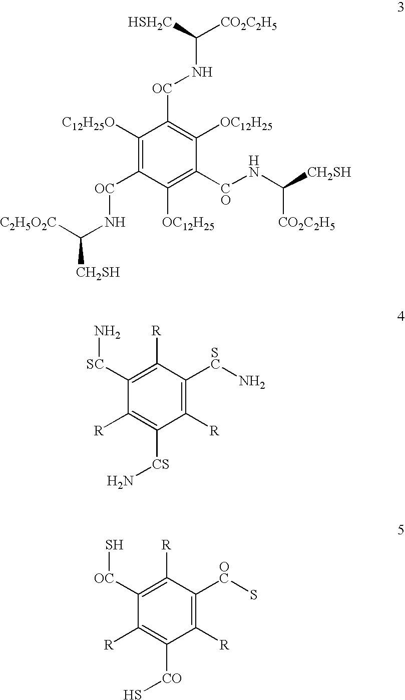

Structures of surface templates 3-5.

Shown in Scheme 2 below is the synthesis of the sulfinamides 2 of this invention. As shown in the Scheme, the sulfinamides were obtained in enantiomerically pure form by amination of a single diastereomer of the menthol ester of the chiral sulfinic acid. Either enantiomer of the sulfinamide could be prepared depending on the chiral enantiomer of menthol used

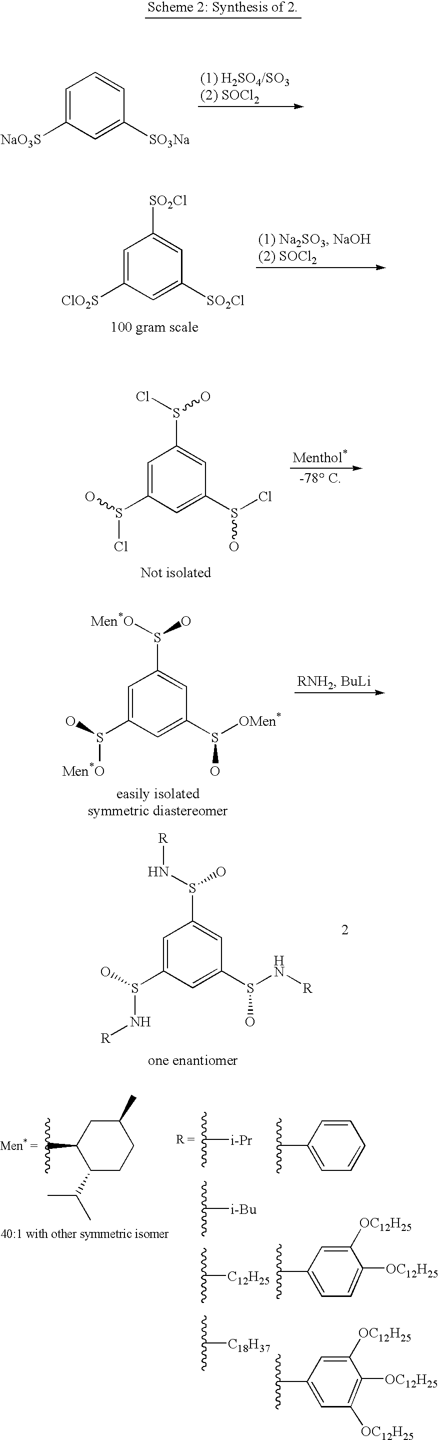

Details of the synthetic procedure are described in Example 2 herein. The symmetric isomer referred to is the compound with all of the sulfur stereocenters having the same handedness.

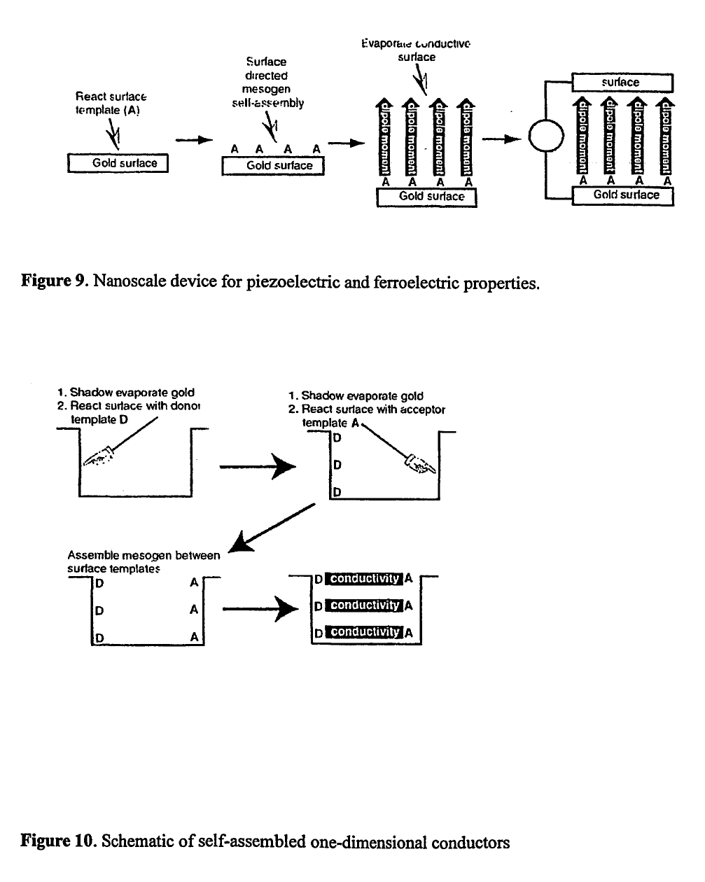

Piezoelectric, ferroelectric, pyroelectric and nonlinear optical devices may be prepared by the method discussed above which is also shown schematically in FIG. 9 and which is further illustrated in Example 4 herein. In an advantageous embodiment of this invention, the molecular array comprises a single isolated columnar structure as previously defined. Scanning probes like electrostatic force microscopy can be used to address an individual column which could then behave as an isolated nanostructural pump, motor, or actuator.

In a still further exemplary embodiment of the method of preparation of a piezoelectric, ferroelectric, pyroelectric, and nonlinear optical device, the surface template compound comprising substituted aromatic rings and the second compound comprising substituted aromatic rings are identical.

Coating the surface template compound onto one of a conductor and semiconductor surface in the methods of preparation of the molecular array and of the piezoelectric, ferroelectric, pyroelectric, and nonlinear optical devices of the invention may be achieved by evaporating a solution comprising the surface template compound onto the surface. Alternatively, the surface may be soaked in a solution of the surface template compound in an organic solvent. Advantageously, the solvent is ethanol and the concentration is between about 0.001 and about 0.01 moles/liter. Another coating method comprises spin-coating or dip-coating the surface template compound comprising substituted aromatic rings using a solution to deposit one molecular monolayer of the surface template compound onto the conductor or semiconductor surface.

Coating the second compound comprising substituted aromatic rings onto the monolayer of the surface template compound in the methods of preparation of the molecular array and of the piezoelectric, ferroelectric, pyroelectric, and nonlinear optical devices of the invention may be achieved by evaporating a solution of the second compound onto the monolayer. Alternatively, the surface comprising the monolayer may be immersed into a solution of the second compound to form the second layer comprising at least one columnar stack of the substituted aromatic rings of the second compound. Another coating method comprises spin-coating or dip-coating the second compound comprising substituted aromatic rings using a solution to deposit onto the surface template monolayer the layer comprising at least one columnar stack of the substituted aromatic rings of the second compound.

One-dimensional conductors may be prepared by the method discussed above which is also shown schematically in FIG. 10 and which is further illustrated in Example 3 herein. Advantageously, one electrode is first coated with a surface template compound which is a hydrogen bond donor and the other is coated with a surface template compound which is a hydrogen bond acceptor. Each electrode may be coated by using one of the procedures described below for the preparation of piezo-, pyro-, and ferro-electric devices and non-linear optical devices. In one embodiment of the method of the invention, the solvent is electrostatically trapped and then the excess washed away.

### EXAMPLES

### Example 1

### Preparation of 1a-d

Preparation of 2,4,6-dodecyloxy-1,3,5-trimethyltricarboxylate. Into a dry 250 mL flask that was outfitted with a stir bar was added 1,3,5-tribromo-2,4,6-tridodecyloxybenzene (4.0 g, 4.6 mmol), and tetrahydrofuran (90 mL). The solution was evacuated and flushed with nitrogen several times and then cooled to −78° C. With vigorous stirring, a t-butyllithium solution (25 mL, 1.5 M solution in hexanes) was added dropwise. After the solution had stirred at −78° C. for 3 hours it became intensely yellow. At −78° C., the reaction was quenched by the addition of methyl chloroformate (3.6 mL, 46 mmol) at a medium pace. The mixture was warmed to room temperature and allowed to stir for ca. 12 h. H₂O (100 mL) was added to the solution and then extracted three times with diethyl ether (100 mL each). The organic phases were combined and dried over sodium sulfate followed by silica gel chromatography (2% Et₂O/hexanes) light-yellow liquid (1.1 g, 1.3 mmol, 29%).

2,4,6-tridodecyloxy-1,3,5-benzenetricarbonyl trichloride. In a 250 ml flask outfitted with a reflux condenser and a magnetic stir bar was added 2,4,6-dodecyloxy-1,3,5-trimethyltricarboxylate (1.0 g, 1.2 mmol) and sodium hydroxide (4.97 g, 124.2 mmol) followed by isopropanol (38 mL) and then H₂O (19 mL). The mixture was heated under reflux (100° C. oil bath) for 12 hours, cooled to room temperature, and concentrated under reduced pressure. On ice, an HCl solution (1 N, 50 mL) was added to the gummy residue and made slightly acidic by addition of a concentrated HCl solution. After extracting this solution three times with Et₂O (50 mL each), the organic solutions were combined, washed with brine (100 mL), and dried over sodium sulfate. Concentration under reduced pressure yielded an off-white foam. To the flask containing the triacid was immediately added CH₂Cl₂ (40 mL) and 0.9 mL SOCl₂ (1.4 g, 11.8 mmol) under an inert atmosphere. The reaction mixture was heated at reflux for 2 h and the volatiles were removed by distillation under reduced pressure, yielding a viscous oil (0.94 g, 1.2 mmol, quantitative).

Synthesis of 1a-d. To a 5 mL flask with a magnetic stirbar under a nitrogen atmosphere was added sequentially 2,4,6-tridodecyloxy-1,3,5-benzenetricarbonyl trichloride (0.11 g, 0.13 mmol), CH₂Cl₂ (1.5 mL), Et₃N [62 μL, 0.4 mmol (twice as much if the amine hydrochloride was used)], and the amine or amine hydiochloride (0.4 mmol). After stirring for 2 h, the mixture was diluted with CH₂Cl₂ (20 mL) and NaHCO₃ (20 mL, sat. aqueous). The phases were separated and the aqueous one extracted twice with CH₂Cl₂ (20 mL each). The organic phases were combined, dried over sodium sulfate, concentrated under reduced pressure. The solids may be either recrystallized from methanol to afford white solids or chromatographed on silica gel with a gradient elution (30% to 45% diethyl ether/hexanes).

### Example 2

### Preparation of 2

Benzene-1,3,5-Trisulfonyl Chloride. Sodium benzene-1,3,5-trisulfonate (15 g, 39 mmol, 1 eq.) was added into of SO₂Cl₂ (70 ml, 25 eq.), then dimethyl formamide (9 ml, 3 eq.) was added carefully. The reaction mixture was refluxed for 3 hours. After cooling down to room temperature, the reaction mixture was poured into 500 ml of crashed ice with vigorously stirring. The solid was filtered and washed with water. The solid was dried in vacuum at the presence of NaOH and anhydrous CaSO₄. Slightly red powder (11.155 g, yield 76%) was obtained, which was pure enough to be used in the next step. Crystals can be obtained after recrystallization from ethyl acetate.

Sodium Benzene-1,3,5Trisulfinate. Benzene-1,3,5-trisulfonyl chloride (11.15 g, 30 mmol, 1 eq.) was added into a solution of Na₂SO₃ (34 g, 9 eq.) in 90 ml water at 75-80° C. portionwise. During the addition, a solution of NaOH (7.2 g, 6 eq.) in 15 ml of water was added to keep the PH at 9 to 10. The mixture was stirred at 75-80° C. for 4 hours. After water was evaporated, 200 ml of acetone were added. After being filtered and washed with more acetone, the solid was dried in the oven at 120° C. overnight. The crude product was used in the next step without the removal of inorganic salts.

Benzene-1,3,5-Trimenthyl Sulfinate. To a solution of 18-crown-6 (1.588 g, 0.6 eq.) and SO₂Cl₂ (18 ml,25 eq.) in 80 ml of CH₂Cl₂ was added crude sodium benzene-1,3,5-trisulfinate (15 g, 1 eq containing 10 mmol benzene-1,3,5-trisulfinate as calculated) portionwise. The mixture was stirred at room temperature under a nitrogen atmosphere overnight. After the unreacted solid was filtered, CH₂C1₂ and SO₂Cl₂ were evaporated. The crude benzene-1,3,5-trisulfinyl chloride (crown ether was contained) was obtained as viscous oil quantitatively. To a solution of crude benzene-1,3,5-trisulfinyl chloride in 120 ml of CH₂Cl₂ was added a solution of (−) Menthol (4.688 g, 30 mmol) and ⁱPr₂NEt (5.75 ml, 33 mmol) in 60 ml of CH₂Cl₂ dropwise at-78° C. over 0.5 hour. The mixture was stirred at −78° C. for 1 hour. After CH₂Cl₂ was evaporated, 100 ml ethyl acetate was added. The solution of crude product in ethyl acetate was washed with 1N HCl, 2% NaHCO₃, and brine subsequently, then dried with anhydrous Na₂SO₄. The crude product was purified by flash chromatography twice. A mixture of ether and CH₂Cl₂ (1:30) was used as eluent in the first chromatography and benzene-1,3,5-trimenthyl sulfinate was obtained as a mixture of 4 diastereomers in 61% yield from benzene-1,3,5-trisulfonyl chloride. A mixture of ethyl acetate and hexanes (1:10) was used as eluent in the second chromatography, and the diastereomers were partially isolated. The homochiral benzene-1,3,5-trisulfinates ((R,R,R)&(S,S,S)) were obtained as viscous oil in the ratio of 1:38 and the yield of 7% from benzene-1,3,5-trisulfonyl chloride.

Benzene-1,3,5-Trialkyl Sulfinamide. General Procedure: To a solution of primary amine (4.5 mmol) in 30 ml of THF was added a solution of nBuLi in hexane (1.6 M, 2.6 ml, 4.2 mmol) under nitrogen at 0° C. After being stirred under nitrogen at 0° C. for 15 minutes, to the mixture was added a solution of benzene-1,3,5-trisulfinate (0.1M, 3 ml, 0.3 mmol) under a nitrogen atmosphere at 0° C. After the reaction was complete (benzene-1,3,5-trisulfinate disappeared on TLC), 2 ml brine was added. The organic layer was separated and dried with anhydrous Na₂SO₄. The pure sulfinamide was obtained as white powder in the yield from 50% to 70% after recrystallized from proper solvents.

### Example 3

### Preparation of a Conductor From the Trialkylamide or Sulfinamide Compounds

A solution of the compound is prepared and two parallel surfaces are placed in the solution. Each surface is an insulator that is shadow evaporated with gold. An electric field (c.a. 30V) is then placed between the two surfaces, whereupon the columnar stacks self-assemble to form the conductor.

Alternatively in the experiment above, each of the surfaces may be coated with a surface hydrogen bond donor template and a surface hydrogen bond acceptor template, as shown in FIG. 10, where A is a hydrogen bond donor and B is a hydrogen bond acceptor. The dipole moments of the molecules in solution align themselves to allow hydrogen bonding with the surface template molecules, whereupon the columnar stacks self-assemble to form the conductor.

Alternatively in the experiment above, each surface can also be silicon or doped silicon. Other conductors besides gold may also be used.

### Example 4

### Preparation of Piezoelectric, Ferroelectric, Pyroelectric, and Non-Linear Optical Devices From the Trialkylamide or Sulfinamide Compounds

A surface as described in experiment 3 is coated with surface template molecules. Evaporation from solution is then used to assemble molecules atop the templates. This creates a polar stack. Each stack can then be addressed with an electromotive force microscope to show electrostriction and ferroelectricity. By irradiating these stacks with laser light the molecule can generate a frequency doubled signal-second harmonic generation.

Alternatively in the experiment above, the molecules are assembled atop the templates by Langmuir-Blodgett film transfer.

It should be understood that various changes and modifications to the preferred embodiments described herein will be apparent to those skilled in the art. Such changes and modifications can be made without departing from the spirit and scope of this invention and without diminishing its attendant advantages. The scope of the invention is covered by the appended claims.

1. A three-dimensional molecular array formed on one of a conductor and semiconductor surface, comprising at least one columnar stack comprising a plurality of substituted aromatic rings, wherein the aromatic rings of each columnar stack lie about parallel to the surface, and the columnar stack comprises a plurality of hydrogen bonds between substituents of different rings.

2. The array of claim 1, wherein the array is a one-dimensional conductor having conductance in a direction about perpendicular to the surface.

3. The array of claim 1, wherein the columnar stack is chiral.

4. The array of claim 1, wherein the aromatic rings are not covalently bound to each other.

5. The array of claim 4, wherein each aromatic ring comprises a first substituent in each of the 2, 4, and 6 positions of the ring, wherein the first substituent is not hydrogen, and a second substituent in each of the 1, 3, and 5 positions of the ring, and the second substituent is selected from the group consisting of a carboxylic group and a group having the structural formula 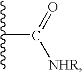
wherein R is selected from the group consisting of hydroxy, substituted and unsubstituted alkoxy, substituted and unsubstituted alkyl, substituted and unsubstituted aryl, substituted and unsubstituted acyl, and 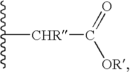
wherein R″ is selected from the group consisting of hydrogen, a substituted alkyl and an unsubstituted alkyl, and R′ is selected from the group consisting of hydrogen, a substituted alkyl and an unsubstituted alkyl.

6. The array of claim 5, wherein the first substituent is n-dodecyloxy, and the second substituent is selected from the group consisting of substituents I-IX:

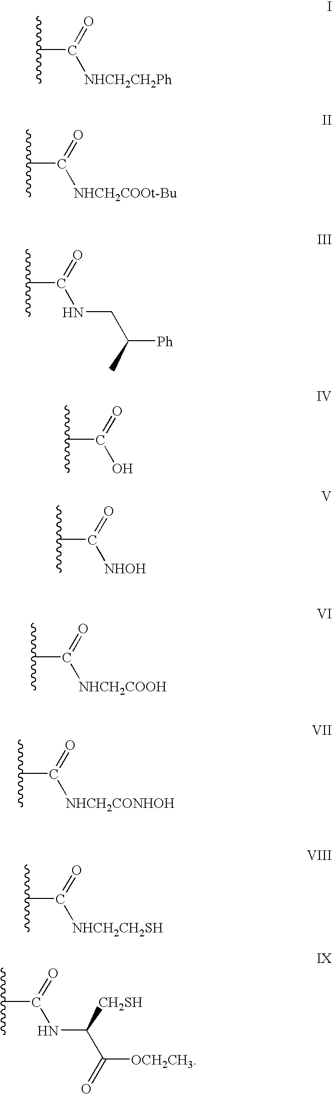

7. The array of claim 4, wherein each aromatic ring comprises a sulfinamide substituent in each of the 1, 3, and 5 positions of the ring, wherein the nitrogen atom in each sulfinamide substituent is bound to at least one hydrogen atom.

8. The array of claim 7, wherein each sulfinamide substituent has the same chiral configuration.

9. The array of claim 7, wherein each sulfinamide substituent is selected from the group consisting of substituents X-XV and their enantiomers:

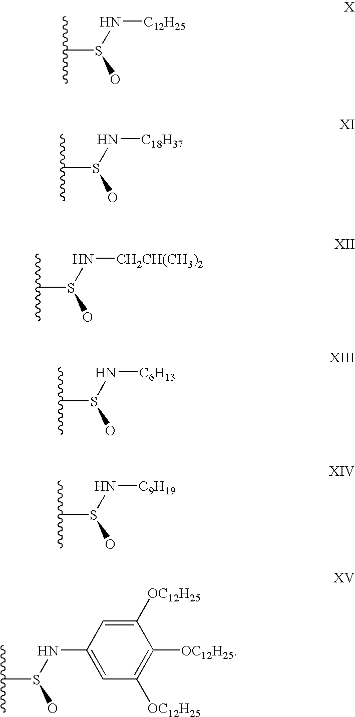

10. The array of claim 1, wherein the 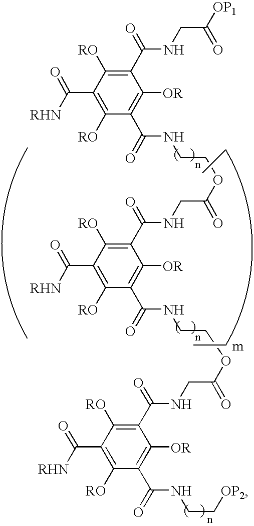
wherein n is an integer from 5 to 10 and m is an integer from 5 to 15.

11. The array of claim 1, wherein the conductor surface is selected from the group consisting of a metal surface, a metal oxide surface, and a graphite surface.

12. The array of claim 1, wherein the semiconductor surface is a silicon surface.

13. The array of claim 1, wherein the semiconductor surface is a doped semiconductor surface.

14. A single one molecule wide columnar stack formed on one of a conductor and semiconductor surface, wherein the columnar stack comprises a plurality of substituted aromatic rings, and the aromatic rings of each columnar stack lie about parallel to the surface, and the columnar stack comprises a plurality of hydrogen bonds between substituents of different rings.

15. The columnar stack of claim 14, wherein the stack is a one-dimensional conductor having conductance in a direction about perpendicular to the surface.

16. The columnar stack of claim 14, wherein the columnar stack is chiral.

17. The columnar stack of claim 14, wherein the aromatic rings are not covalently bound to each other.

18. The columnar stack of claim 14, wherein the semiconductor surface is a doped semiconductor surface.

19. A method for the preparation of a three-dimensional molecular array on one of a conductor and semiconductor surface, the method comprising:
(a) coating onto the surface a surface template compound comprising substituted aromatic rings to form a surface template molecular monolayer; and
(b) coating onto the surface template monolayer a second compound comprising substituted aromatic rings to form at least one columnar stack comprising the substituted aromatic rings of the second compound, wherein the aromatic rings of each columnar stack and of the surface template molecules lie about parallel to the surface, and the columnar stack comprises a plurality of hydrogen bonds between substituents of different rings.

20. The method of claim 19, wherein the surface template compound comprising substituted aromatic rings and the second compound comprising substituted aromatic rings are identical.

21. The method of claim 19, wherein the array is a one-dimensional conductor having conductance in a direction about perpendicular to the surface.

22. The method of claim 19, wherein the columnar stack is chiral.

23. The method of claim 19, wherein the aromatic rings are not covalently bound to each other.

24. The method of claim 19, wherein the semiconductor surface is a doped semiconductor surface.

25. A method for the preparation of at least one of a piezoelectric, ferroelectric, pyroelectric, and non-linear optical device on one of a conductor and semiconductor surface, comprising:
(a) coating a surface template compound comprising substituted aromatic rings onto the surface to form a surface template molecular monolayer;
(b) rinsing and drying the surface;
(c) coating onto the monolayer a second compound comprising substituted aromatic rings to form a second layer comprising at least one columnar stack of the aromatic rings of the second compound, wherein the aromatic rings of each columnar stack and of the surface template molecules lie about parallel to the surface, and the columnar stack comprises a plurality of hydrogen bonds between substituents of different rings; and
(d) evaporating onto the second layer another conductive layer so as to provide another conducting surface in contact with the second layer.

26. A method for the preparation of a one-dimensional conductor on one of a conductor and semiconductor surface, the one-dimensional conductor having conductance in a direction about perpendicular to the surface, the method comprising
(a) providing a solution of a compound comprising a substituted aromatic ring;
(b) providing two electrodes having about parallel surfaces in contact with the solution, wherein each electrode is one of a conductor and a semiconductor; and
(c) applying an electric potential between the electrodes, thereby forming a plurality of columnar stacks each comprising a plurality of substituted aromatic rings, wherein the aromatic rings of each columnar stack lie about parallel to the surface of each electrode, and the columnar stack comprises a plurality of hydrogen bonds between substituents of different rings, and wherein each one of the stacks has a respective dipole moment aligned between the two electrodes.

27. A method for the preparation of a one-dimensional conductor, the one-dimensional conductor having conductance in a direction about perpendicular to the surface, the method comprising
(a) providing a solution of a compound comprising a substituted aromatic ring;
(b) coating a surface template hydrogen bond donor compound onto a first one of a conductor and a semiconductor surface to form a first molecular monolayer;
(c) coating a surface template hydrogen bond acceptor compound onto a second one of a conductor and a semiconductor surface to form a second molecular monolayer; and
(d) contacting the first and second surfaces with the solution, wherein the first and second molecular monolayer are about parallel and face each other, thereby forming a plurality of columnar stacks each comprising a plurality of substituted aromatic rings, wherein the aromatic rings of each columnar stack lie about parallel to the first one of the conductor and semiconductor surface and about parallel to the second one of the conductor and semiconductor surface, and the columnar stack comprises a plurality of hydrogen bonds between substituents of different rings, and wherein each one of the stacks has a respective dipole moment aligned between the two surfaces.

28. A compound comprising an aromatic ring, the aromatic ring comprising an n-dodecyloxy substituent in each of the 2, 4, and 6 positions of the ring and a second substituent in each of the 1, 3, and 5 positions of the ring, wherein the second substituent is selected from the group consisting of a carboxylic group and a group having the structural formula 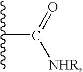
wherein R is selected from the group consisting of hydroxy, substituted and unsubstituted alkoxy, substituted and unsubstituted alkyl, substituted and unsubstituted aryl, substituted and unsubstituted acyl, and 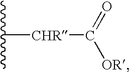
wherein R″ is selected from the group consisting of hydrogen, a substituted alkyl and an unsubstituted alkyl and R′ is selected from the group consisting of a substituted alkyl and an unsubstituted aLkyl.

29. The compound of claim 28, wherein the second substituent is selected from the group consisting of structures I-IX:

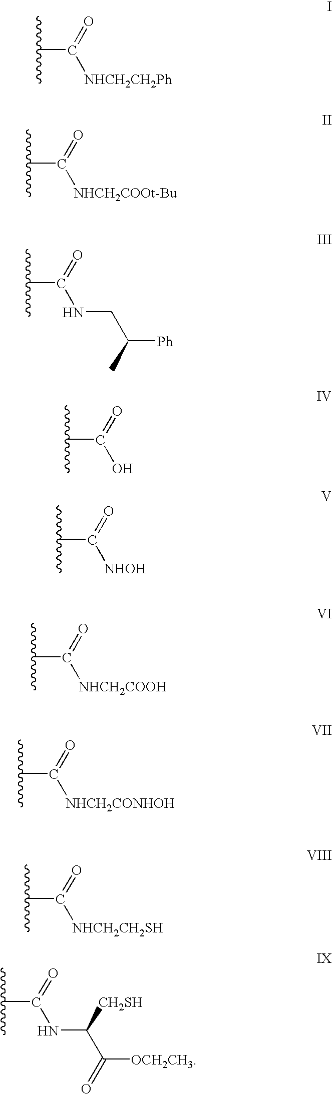

30. A compound comprising an aromatic ring, the aromatic ring comprising a sulfinamide substituent in each of the 1, 3, and 5 positions of the ring, wherein the nitrogen atom in each sulfinamide substituent is bound to at least one hydrogen atom.

31. The compound of claim 30, wherein each sulfinamide substituent has the same chiral configuration.

32. The compound of claim 30, wherein the sulfinamide substituent is selected from the group consisting of structures X-XV and their enantiomers: [chemical structure]
